(19) United States
(12) Patent Application Publication
(10) Pub. No.: US 2006/0025732 A1
(43) Pub. Date: Feb. 2, 2006

(54) Sanitary article for revealing and observating personally physiological condition

(75) Inventors: Nai-Ko Ying, Jhonghe City (TW); Jeng-Horng Hou, Jhonghe City (TW); Chia-Lin Hsieh, Jhonghe City (TW)

Correspondence Address: HDSL, 4331 STEVENS BATTLE LANE, FAIRFAX, VA, 22033, US

(73) Assignee: Neter Marketing Co., Ltd.

(21) Appl. No.: 11/168,474

(22) Filed: Jun. 29, 2005

(30) Foreign Application Priority Data
Jul. 29, 2004 (TW) 093122794

(51) Int. Cl. A61F 13/15

(52) U.S. Cl. 604/361

(57) ABSTRACT

A novel design and usage of sanitary article for advising personally physiological condition includes a detection zone or testing zone, a coating strip or polymer material, which is imbedded into and disposed on the sanitary article, to reveal the assigned physiological advisement. The secretions or menses remained on the sanitary article reacts with the detection zone or testing zone and displays the health condition of the user thereon. Accordingly, the user of the sanitary article can advice personally health condition as preventative diagnostics.

### BACKGROUND OF THE INVENTION

The present invention relates generally to a sanitary article and a method for advising personally physiological condition, and more particularly to a sanitary napkin for ladies, a diaper for babies or adults, and a method for performing medical examinations (personal diagnostications).

The most commonly seen sanitary articles, such as diapers for babies or adults, or sanitary napkins for ladies, wraps the lower portion of a human body to prevent urine, excrement or menses from leaking away. The function and mechanism of the sanitary articles is to reduce the load of cleaning the waste product of children or elderly and to absorb secretions during the menses period of a lady.

However, in regard to healthcare point of view, taking advantage of the human secretions (urine, excrement, or menses), to examine body physiological conditions, are very convenience and practical. For example, diagnostic target could be pH value, leukocyte, nitrites, proteins, glucose, ketones, urobilinogen, bilirubin, hemoglobin, stool urobilinogen, microbiology, etc. In this manner, abnormality of health condition can be observed by customers (revealed outcome) and thus can be treated in an earlier stage through such alarming and advice, thereby preventing such abnormality from developing into severe diseases.

In general, conventional sanitary articles are provided for preventing leakage of waste products, which can not perform any medical examination and advisement. Nevertheless, once such periodical health examination is inadvertently missed, it is likely that a small health problem may develop into an immedicable disease.

Consequently, the inventors of the present invention has devoted themselves based on expertise in both of technology biomedical technology and behavior science to the development of a new sanitary article that can foresee the problems as set forth above.

### BRIEF SUMMARY OF THE INVENTION

The present invention is primarily to provide a sanitary article and method for advising physiological condition. The designed sanitary article contains coating strip or polymer test materials, to perform the desired physiological examinations. The secretions or menses remained on the sanitary article, then reacts with the test materials and reflects the health condition of the user thereon. Thus, this present invention, being advising tools, provides the early call of healthcare plan.

Accordingly, the components of sanitary article of the present invention includes an absorbing body having a liquid absorbing core, and testing zone or detection zone is disposed on the liquid absorbing core for examining secretion of a human body, whereby health condition of the human body is examined and monitored by colorimetrical charge onto testing zone.

The method for advising the physiological examinations of the present invention includes the following steps. First, a sanitary article is provided, which contains a testing zone (detection zone) disposed thereon. Then, secretion of a human body is absorbed into the testing zone of the sanitary article. Next, the coating strip or polymer reacts with the secretion right away. Finally, the personally physiological advisement results are displayed onto the testing zone for further action plan.

### ILLUSTRATING OF THE INVENTION

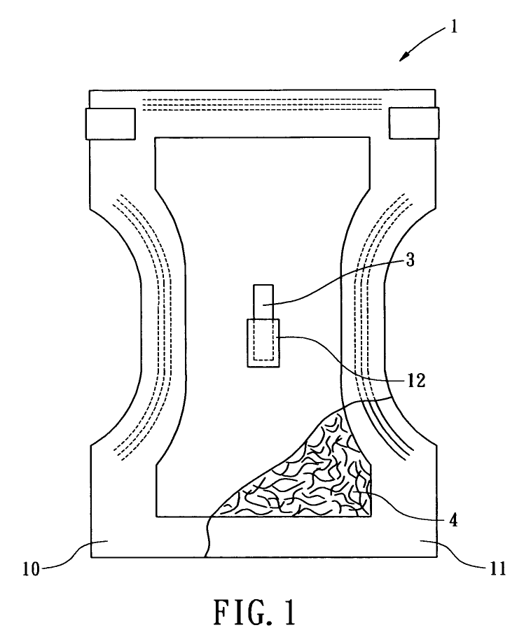

FIG. 1 is a diaper, illustrating a sanitary article for advising personal physiological condition, in accordance with one embodiment of the present invention.

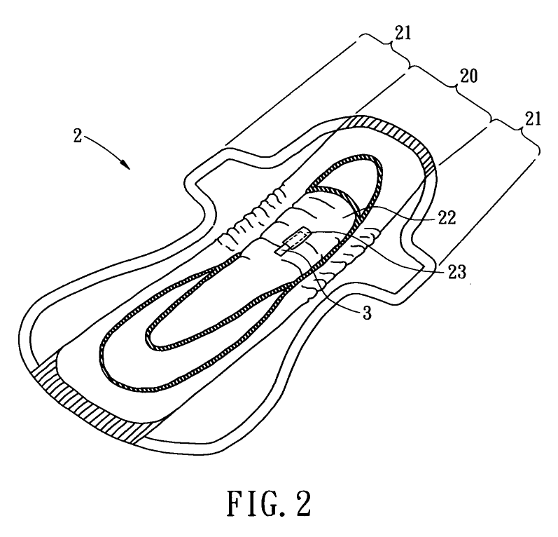

FIG. 2 is a sanitary napkin, illustrating a sanitary article for advising personal physiological condition, in accordance with another embodiment of the present invention.

### DETAILED DESCRIPTION OF THE INVENTION

In order to better understanding the features and technical contents of the present invention, the present invention is hereinafter described in detail by incorporating with the accompanying illustration. However, the accompanying illustration are only for the convenience of description, no limitation is intended thereto.

The present invention provides a sanitary article for advising personal physiological condition, such as a diaper for babies or adults, or sanitary napkins for ladies. The sanitary article wraps the lower portion of a human body to prevent urine, excrement or menses from leaking away. Meanwhile, it can also reveal the health condition of a human body.

Referring to FIG. 1, a sanitary article in accordance with one embodiment of the present invention is illustrated. The sanitary article in this particular embodiment is a diaper for babies and elders. The sanitary article 1 includes an osmolar front layer 10, a non-osmolar back layer 11, and an absorbing core layer 12 sandwiched between the front layer 10 and the back layer 11. The upper and lower portions of the sanitary article 1 are wider in width than that of the central portion of the sanitary article 1, one can thus wear the sanitary article 1 covering the lower portion of ones body. Since the structures of the sanitary article 1 shown in FIG. 1 is similar to that of a conventional sanitary article, detailed description of which is thus neglected.

The detection or testing zone 3 is disposed on the top portion of the sanitary article 2 that contacts human body. The detection or testing zone 3 performs the observation of desired personal physiological condition through absorbing the secretion, such as urine, excrements or menses. The detection or testing zone 3 is coating a strip or polymer materials. The detection or testing zone 3 can be disposed directly on the front layer 10 of the sanitary article 1, or be inserted into an osmolar bag 13 being disposed on the front layer 10. In addition, the detection or testing zone 3 can also be sandwiched between the front layer 10 and the absorbing core layer 12, so long as the detection or testing zone 3 can absorb the secretion, such as urine, excrement or menses, of the user.

In this manner, when the detection or testing zone 3 contacts and absorbs the secretions of the user, the detection or testing zone 3 can react to the absorbed secretion and shows a result of physiological condition. Once any abnormality occurs, the user can consult a doctor to his or her soonest convenience, thereby preventing such abnormality from being delayed from proper treatments.

Referring to FIG. 2, a sanitary article in accordance with another embodiment of the present invention is illustrated. The sanitary article in this particular embodiment is a sanitary napkin for ladies. The sanitary article 2 includes an extended absorbing body 20, and two wing portions 21 disposed on the left and right sides of the body. The body 20 further includes a liquid absorbing core 22. In this particular embodiment, a detection or testing zone 3 is disposed on the top portion of the sanitary article 2 that contacts human body. A detection or testing zone 3 can be disposed directly on the liquid absorbing core 22 of the body 20 of the sanitary article 2. On the other hand, a liquid absorbing bag 23 can be disposed on the liquid absorbing core 22 for inserting the examination member 3 into the bag 23. In addition, the detection zone or testing zone 3 can also be embedded into the liquid absorbing core 22, so long as the detection zone or testing zone 3 can absorb the secretion of the user. Thus, during the menses period of a lady, the secretion is absorbed into the sanitary article 2 and the detection zone or testing zone 3 then. The detection zone or testing zone 3 then reacts to the absorbed secretion and shows a result of physiological condition.

In addition, the sanitary article of the present invention is also applicable to condom, mouth mask and etc.

Therefore, the present invention contains a sanitary article that can provide medical or physiological information through biomedical technologies. Since sanitary articles are used regularly or periodically, the user of which can properly monitor his or her health condition. Consequently, early health problems can be treated without being delayed and developed into an immedicable disease due to delay of treatment or ignorance of alarming.

In summary, the sanitary article of the present invention indeed contains patentable subject matter that is consistent with the utility, novelty and non-obviousness requirements as set forth in the patent law. Therefore, a grant of letters patent for the present invention is respectively requested.

Since, any person having ordinary skill in the art may readily find various equivalent alterations or modifications in light of the features as disclosed above, it is appreciated that the scope of the present invention is defined in the following claims. Therefore, all such equivalent alterations or modifications without departing from the subject matter as set forth in the following claims is considered within the spirit and scope of the present invention.

What is claimed is:

1. A physiological advising device, comprising:
sanitary article covering the lower portion of a human body to prevent secretion or body fluid from leaking away; and
detection zone or testing zone disposed on a portion of the sanitary article contacting a human body for examining secretion or body fluid of the human body,
whereby health condition of the human body is examined and monitored.

2. The device as recited in claim 1, wherein the sanitary article is a diaper.

3. The device as recited in claim 2, wherein the diaper comprises disposable osmolar layers for inserting and displaying the detection zone or testing zone therein.

4. The device as recited in claim 3, wherein the detection zone or testing zone is sandwiched between the front layer and the absorbing core layer.

5. The device as recited in claim 1, wherein the detection zone or testing zone is disposed in the absorbing core layer.

6. The device as recited in claim 1, wherein the sanitary article is a sanitary napkin.

7. The device as recited in claim 6, wherein the sanitary napkin comprises an extended absorbing body with a liquid absorbing core, and two wing portions disposed on the left and right sides of the extended absorbing body.

8. The device as recited in claim 6, wherein a liquid absorbing bag is formed on the liquid absorbing core for inserting and displaying the detection zone or testing zone therein.

9. The device as recited in claim 1 wherein the detection zone or testing zone comprises a test paper or a test piece.

10. A method for advising personally physiological condition, comprising the steps of:
providing a sanitary article having a detection zone or testing zone disposed thereon;
absorbing secretion of a human body by using the detection zone or testing zone of the sanitary article;
allowing the detection zone or testing zone to react with the absorbed secretion; and
displaying physiological results on the detection zone or testing zone.
